(19) United States
(12) Patent Application Publication
(10) Pub. No.: US 2009/0018522 A1
(43) Pub. Date: Jan. 15, 2009

(54) TISSUE MODIFICATION BY TARGETED DELIVERY OF HEAT

(75) Inventors: DAVID WEINTRAUB, Yavne (IL); YORAM ESHEL, TEL AVIV (IL)

Correspondence Address: CANTOR COLBURN, LLP, 20 Church Street, 22nd Floor, Hartford, CT, 06103, US

(73) Assignee: ANIMA VENTURES LTD.

(21) Appl. No.: 11/775,582

(22) Filed: Jul. 10, 2007

(51) Int. Cl. A61M 31/00; A61B 18/04; A61F 7/12

(52) U.S. Cl. 604/506; 604/113; 606/27; 607/113

(57) ABSTRACT

A method for manipulating a tissue by targeted delivery of heat by heating a liquid contained in an injecting means to a predetermined temperature level and injecting a predetermined volume of said heated liquid into a predetermined depth of the tissue through a needle attached to the injecting means, to thereby manipulate the tissue in the injected area. An apparatus adapted for manipulating a tissue by targeted delivery of heat having a heating means adapted to heat a liquid contained in an injecting means to a predetermined temperature level; an injecting means adapted to contain liquid to be injected said tissue and to inject a predetermined volume of said liquid into a predetermined depth of the tissue through a needle; a temperature sensor adapted to identify the temperature level of the liquid contained in the injecting means and to produce a signal indicating said temperature level; a controller adapted to monitor the sensor signal and to activate or deactivate said heating means and an electromechanical element according to the temperature sensed, and an electromechanical element either integrated or non-integrated to the apparatus that is adapted to allow the injection of the heated liquid.

### FIELD OF THE INVENTION

The present invention relates to apparatus and methodology for tissue manipulation by targeted delivery of heat in general, and to manipulation of a tissue by targeted injection of heated liquid in particular.

### BACKGROUND

### General

Medical aesthetic treatments for the face and the body have become very popular in recent years. More and more people are aware of their appearance and pursuing eternal youth. Parallel to the growing awareness, the availability of new treatments, devices and medicaments for such treatments have grown tremendously. Fat reduction, cellulite refinement, skin tightening, wrinkle elimination, stretch marks improvement, scar improvement, hair removal, hair re-growth, acne treatment as well as other treatment have become accessible to the public, less expensive, more friendly and most importantly, minimally invasive and with almost no downtime.

### Mesoplasty

The Mesotherapy discipline involves painless microinjections into the skin achieving a desired therapeutic result. Although Mesotherapy has been used extensively in Europe, it has only recently been introduced in the United States. Uses of Mesotherapy include fat and cellulite reduction, control of pain conditions, hair re-growth, weight loss, wrinkle elimination, scar improvement, acne treatment, arthritis and joint problems, sports injuries, stretch marks, among others. Treatment with Mesotherapy for aesthetic/cosmetic improvement is referred to as Mesoplasty®. Mesotherapy typically consists of numerous injections of variable depth delivering the desired medication into different layers of the skin and the subcutaneous tissue. In one session the patient may receive between a hundred to several hundred injections, depending on the technique used and the treated area.

In contrast to traditional medicine that typically uses oral, intramuscular or intravenous routes to deliver pharmaceuticals to patients and thus resulting in systemic effects as well as systemic side effects, the mesotherapy technique focuses on local delivery in order to achieve local results and minimize systemic effects. By administering minute amounts with each injection and by delivering the medication directly to the target area mesotherapy minimizes both local and systemic side effects while amplifying the results.

However, the major drawback of the Mesoplasty discipline is the usage of medication. Medications are expensive, and bare an inherent risk for the user. In addition, although the targeted delivery of the medications reduces the side effects, still, undesired effect may occur. The present invention is aiming to solve this problem by injection of an inert liquid that is favorable to the human body and is almost costless.

### Mesotherapy Gun

The medications used in Mesoplasty® can be administered via a specially designed injector, know as Mesotherapy gun, Mesogun, injection gun, pistolets or automatic injector. The Mesogun delivers the medicine painlessly into the skin. The delivery system uses a very fine needle that moves at a high rate of speed leading to painless treatment. like mesotherapy, the injection devices used in mesotherapy have also gone through many generations of evolution. Modern injector devices range from fully automatic to semi-automatic devices. Functions often include continuous versus intermittent injection, variability of rate of injection or frequency of the intermittent mode, variability of depth of injection and capability of the device to accommodate various syringe sizes. EP1208858 discloses a mesotherapy injector device for medical and cosmetic treatment by successive injections with a disposable syringe comprising a pneumatic means to alternately translate the syringe plunger. Additional specific example of injector device is disclosed in U.S. Pat. No. 4,790,823.

### Hyperthermia Therapy for Cancer Treatment

Hyperthermia therapy is a treatment in which body tissue is exposed to high temperatures to damage and kill cancer cells, or to make cancer cells more sensitive to the effects of radiation and certain anticancer drugs. Local hyperthermia treatment (heat applied to a very small area, such as a tumor) is a well-established cancer treatment method based on the fact that when applying a temperature of 106Â° F. for one hour within a cancer tumor, destroys the cancer cells.

Hyperthermia therapy is almost always used with other forms of therapy such as radiation therapy, chemotherapy, and biological therapy, in order to increase their effectiveness. With hyperthermia therapy the area may be heated externally with high-frequency waves aimed at a tumor from a device outside the body. To achieve internal heating, one of several types of sterile probes may be used, including thin, heated wires or hollow tubes filled with warm water; implanted microwave antennae; and radiofrequency electrodes. One other technique uses ultra-high frequency sound waves to produce heat within the tumor, since ultrasound is more easily focused than other energy modalities.

The usage of heat for treating cancer according to any of the techniques available today is awkward, expensive, require complicated equipment and very uncomfortable to the treated person. The present invention is aiming to provide a simple, cheap and friendly apparatus for destruction of cancer cells and necrosis induction, and methods for using said apparatus.

### Application of Heat for Aesthetic Applications

The use of heat to tighten and renew the skin's collagen is well known in the art. The exposure to heat encourages the natural mechanisms of the body to build new collagen resulting in immediate tightening and improved tone of the skin in the treated area. U.S. Pat. No. 5,458,596 discloses an apparatus and method for control contraction of tissue that includes collagen fibers. The apparatus includes an electrode adapted to be introduced through an operating cannula; the electrode delivers a controlled amount of energy for heating the tissue in order to achieve contraction of the collagen fibers. In U.S. Pat. No. 5,507,790 a method for non-invasive reduction of subcutaneous fat tissue by focusing radiant energy to raise the temperature to range of about 10 to 41.5° for accelerating lypolisis is disclosed. The method may be augmented with topically applied lypolitic augmentation drugs, and the radiant energy according to the disclosed invention may be ultrasound, RF or microwave energy. U.S. Pat. No. 5,778,894 discloses a method for treating cellulite involving deep heating of the cellulite areas by applying electromagnetic energy in a pulsed manner, while U.S. Application No. 2004/0073079 discloses a method and apparatus for heating tissue in region of depth by applying optical radiation while cyclically applying cooling to the skin. One another example of a clinically proven technology for tighten and contour the skin by using RF energy is the Thermage technology disclosed in U.S. Pat. No. 5,871,524; U.S. Pat. No. 5,919,219 and U.S. Pat. No. 6,405,090.

The major problems of the methods disclosed in the above are the high occurrence of burns on the treated skin and the high cost of the devices involved in the procedure. The solution provided by the present invention for aesthetic treatments by application of heat is simple, accurate and harmless.

Thus, there is a real need for a reliable, effective, painless, cheap and friendly apparatus for both, the user and the operator.

### SUMMARY OF THE INVENTION

It is the aim of the present invention to provide a novel apparatus and methodology for manipulating a tissue by targeted delivery of heat to the tissue by injecting an inert heated liquid to the treated tissue. There is thus provided in accordance with a preferred embodiment of the present invention a method for manipulating a tissue by targeted delivery of heat, said method comprising the steps of:

a. heating a liquid contained in an injecting means to a predetermined temperature level;

b. directing the injecting means to said tissue; and

c. injecting a predetermined volume of said heated liquid into a predetermined depth of the tissue through a needle attached to said injecting means, to thereby manipulate the tissue in the injected area.

Additionally in accordance with a preferred embodiment of the invention, there is provided a method for manipulating a tissue by targeted delivery of heat comprising the steps of:

a. heating a liquid contained in an injecting means that is functionally connected to an electromechanical element to a predetermined temperature level;

b. directing the injecting means to a target area in said tissue; and

c. activating said electromechanical element to functionally operate the injecting means to inject a predetermined volume of said heated liquid into a predetermined depth of the tissue through a needle attached to said injecting means, to thereby manipulate the tissue in the injected area.

Activation the electromechanical element according to the provided method comprises pressing on the injection trigger. Upon pressing on the injection trigger at least part of the injecting means, a needle attached thereto, or both moves toward the tissue, penetrate it to a certain depth, and inject a predetermined volume of the heated liquid to the tissue.

Further in accordance with a preferred embodiment of the present invention there is provide a method for aesthetic and medical treatment comprising the steps of:

a. heating a liquid contained in an injecting means that is functionally assembled with an electromechanical device to a predetermined temperature level;

b. directing the injecting means to said tissue; and

c. activating the electromechanical device to functionally operate the injecting means and allow the injection of a predetermined volume of said heated liquid into a predetermined depth of the tissue through a needle functionally attached to the injecting means.

The electromechanical device comprises at least an injection trigger adapted to activate an electromechanical element and an electromechanical element.

In accordance with a preferred embodiment of the present invention, the methods above further comprise sensing the temperature of the liquid contained in the injecting means, preferably in a close loop manner, and producing a signal indicating said temperature.

In accordance with an additional preferred embodiment of the present invention, the methods above further comprise deactivating the heating means upon determining that the temperature level of the liquid contained in the injecting means is equal to or above the predetermined temperature level, and activating the heating means upon determining that the temperature level of the liquid contained in the injecting means is below the predetermined temperature level.

Yet, in a further preferred embodiment of the present invention, the methods provided herein further comprise displaying predefined features on a display means. The methods may further comprise producing at least one vocal alert by a speaker means.

Additionally, in accordance with a preferred embodiment of the present invention, there is provided an apparatus adapted for manipulating a tissue by targeted delivery of heat, said apparatus comprises:

A heating means adapted to heat a liquid contained in an injecting means to a predetermined temperature level;

An injecting means adapted to contain liquid to be injected to said tissue and to inject a predetermined volume of said liquid into a predetermined depth of said tissue through a needle;

A temperature sensor adapted to identify the temperature level of the liquid contained in the injecting means and to produce a signal indicating said temperature level;

A controller adapted to monitor the sensor signal and to activate the heating means upon said controller determining that the temperature of the liquid is below the predetermined temperature level or to deactivate the heating means upon said controller determining that the temperature of the liquid is equal to or above the predetermined temperature level.

In a preferred embodiment of the invention, the apparatus further comprise a connector, said connector is functionally connecting the injecting means and the needle, and the heating of the liquid for injection to a predetermined temperature level is performed in the connector.

In additional preferred embodiment, the apparatus provided herein further includes display means adapted to display predefined features. The apparatus may also include a speaker means adapted to produce at least one vocal alert.

The apparatus may also comprise a self test mechanism and/or an auto shut down mechanism.

There is further provided in accordance with a preferred embodiment of the present invention a system for aesthetic and medical treatment of a person in need of such treatment comprising:

An apparatus adapted for manipulating a tissue by targeted delivery of heat into said tissue; and

An electromechanical device adapted for assembling with said apparatus, and allowing the delivery of heat to said tissue according to a predetermined pattern.

Preferably, the apparatus of said system comprises at least a heating means, an injecting means, a temperature sensor and a controller, and the electromechanical device comprises at least an electromechanical element and an injection trigger adapted to activate said electromechanical element.

The system provided herein may further comprise a display means and/or a speaker means.

In accordance with a further preferred embodiment of the present invention there is provided an apparatus adapted for manipulating a tissue by targeted delivery of heat comprising:

A heating means adapted to heat a liquid contained in an injecting means to a predetermined temperature level;

An injecting means adapted to contain liquid to be injected to said tissue and to inject a predetermined volume of said liquid into a predetermined depth of said tissue through a needle;

A temperature sensor adapted to identify the temperature of the liquid contained in the injecting means and to produce a signal indicating said temperature;

A controller adapted to monitor the sensor signal and to activate or deactivate the heating means and an electromechanical element according to the temperature sensed; and

An electromechanical element functionally connected to an injection trigger and to said injecting means and adapted to allow the injection of said heated liquid.

In a preferred embodiment of the invention the apparatus further comprise a connector that is functionally connecting the injecting means and the needle, and the heating of the liquid to a predetermined temperature level is performed in the connector.

In one further preferred embodiment of the invention, the apparatus further comprise a display means and/or a speaker means.

### BRIEF DESCRIPTION OF THE FIGURES

The present invention will be understood and appreciated more fully from the following detailed description, taken in conjunction with the appended figures. Identical structures, elements or parts that appear in more than one figure are generally labeled with a same numeral in all the figures in which they appear. Dimensions of components and features are generally chosen for convenience and clarity of presentation and are not necessarily shown to scale. The figures are listed below:

FIG. 1 is a simplified block diagram illustration of a MesoTherm apparatus in accordance with a preferred embodiment of the invention;

FIG. 2 is a simplified pictorial illustration of a general structure of a MesoTherm apparatus constructed in accordance with a preferred embodiment of the invention, in which the electromechanical element is an integral part of the MesoTherm apparatus;

FIG. 3 is a simplified pictorial illustration of a general structure of a MesoTherm system constructed in accordance with a preferred embodiment of the invention, in which the MesoTherm apparatus does not comprise an electromechanical element as an integral part of it and it is functionally assembled with an independent electromechanical device.

FIG. 4A-C are schematic illustrations of various shapes and locations of the heating means relative to the injecting means. 4A—the heating means is coating the inner wall of the injecting means; 4B—the heating means is located inside the injecting means; 4C—the heating means is located outside the injecting means and it is functionally connecting the injecting means and the needle.

### DETAILED DESCRIPTION OF THE INVENTION

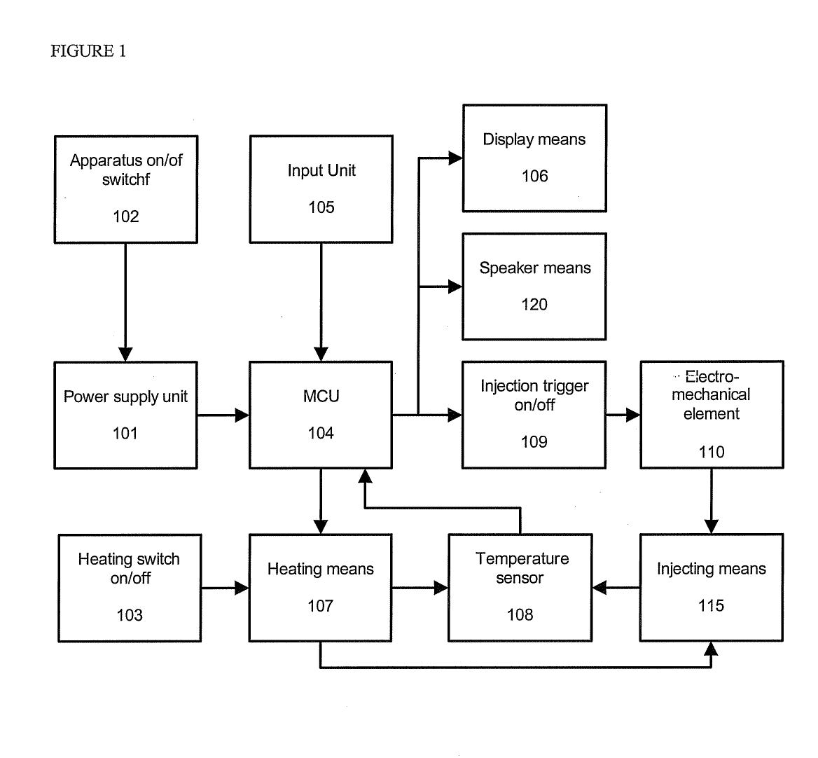

Reference is now made to FIG. 1, which is a simplified block diagram illustration of a MesoTherm apparatus 100 in accordance with a preferred embodiment of the invention. As seen in FIG. 1, the MesoTherm apparatus 100 is adopted for manipulating a tissue by targeted delivery of heat. More specifically, the targeted delivery of heat by the MesoTherm apparatus 100 is conducted by way of targeted injection of an inert liquid to a specific area of a tissue, by controlling, for example, the depth of injection, the injection volume and the temperature of the injected liquid. The term “manipulate”, “manipulating” or “manipulation” of a tissue, as used herein, means applying any desired influence on a tissue. Such influence includes, without limitation: fat reduction, cellulite refinement, body contouring, skin tightening, hair re-growth, wrinkle elimination, scar improvement, acne treatment, stretch marks improvement, destruction of cancer cells and necrosis induction.

The MesoTherm apparatus 100 generally comprises a power supply unit 101, a micro control unit (MCU) 104, an input unit 105, a heating means 107, a temperature sensor 108, an injecting means 115, and optionally an electromechanical element 110, an injection trigger 109, a display means 106 and a speaker means 120.

The power supply unit 101 is controlled by an on/off switch 102 that activates the apparatus or turns it off. In one embodiment of the invention, turning the power supply on activates a self test circle. The power supply unit may comprise an internal battery that makes the apparatus portable and easy to use. In a preferred embodiment of the invention, the battery is a rechargeable battery. The power supply unit 101 may also comprise an electrical power supply that functions both, as the battery charger and as electric supply for the apparatus when the battery is out of charge. In a preferred embodiment the electric power is available for both 110V and 220V currents. In a specific embodiment, the MesoTherm apparatus 100 further comprises a cradle (not shown) that acts as a support when the apparatus is not in use while charging the battery. The power supply unit 101 is functionally connected to a micro control unit (MCU) 104. Predetermined parameters such as the temperature level of the injected liquid, depth of injection, injection volume and injection velocity, are inserted via an input unit 105 and transferred to the MCU 104. The insertion of the predetermined parameters trough the input unit 105 may be performed by selecting a desired value from a predefined menu or by manually entering a chosen parameter. The insertion of the data may be performed mechanically, for example by choosing a preferred value by a selection switch, or it may be performed digitally. The insertion of the data may also be performed via a touch screen, touch pad, a key board or by using any other means know in the art for such purpose that is suitable for the MesoTherm apparatus.

The MCU 104 is functionally connected to a heating means 107 adapted to heat a liquid contained in the injecting means to a predetermined temperature level, and to a temperature sensor 108 adapted to identify the temperature of the liquid in the injecting means and to produce a signal indicating the sensed temperature. The MCU 104 comprises a controller adapted to monitor the temperature sensor 108 signal and to activate the heating means 107 upon said controller determining that the temperature of the liquid is below the predetermined temperature level, or to deactivate the heating means upon determining that the temperature of the liquid is equal to or above the predetermined temperature level. In more details, the controller includes an appropriate control circuitry adapted to receive signals from the temperature sensor 108 and to activate or deactivate the heating means 107 in accordance with the sensed temperature of the liquid contained in the injecting means 115. In accordance with one another embodiment of the invention, the heating means 107 is functionally connected to a temperature on/off switch 103 adapted to enable the activation or deactivation of the heating means. In such embodiment, by turning the temperature switch on, the heating means becomes functionally accessible to receive signals from the MCU 104 and to start heating or to cease heating the liquid contained in the injecting means, depending on the signal obtained. In yet a different embodiment, turning the temperature switch on functionally activates the heating means that begins to heat the liquid contained in the injecting means to a predefined default temperature, which is maintained until a signal from the controller to heat the liquid to a different temperature level is obtained. The default temperature level in accordance with one embodiment of the present invention may be determined as the lowest temperature level that allows manipulation of a tissue according to the methods provided herein. In a preferred embodiment the default temperature level is in the range of 42° C. to 50° C., and even more preferably, the default temperature level is in the range of 45° C. to 48° C. In a further embodiment of the invention, turning the temperature switch 103 on activates a self test circle of the heating means, the temperature sensor or both. The temperature switch 103 may also operate as a safety means that enables an immediate deactivation of the heating means by simply turning the temperature switch off.

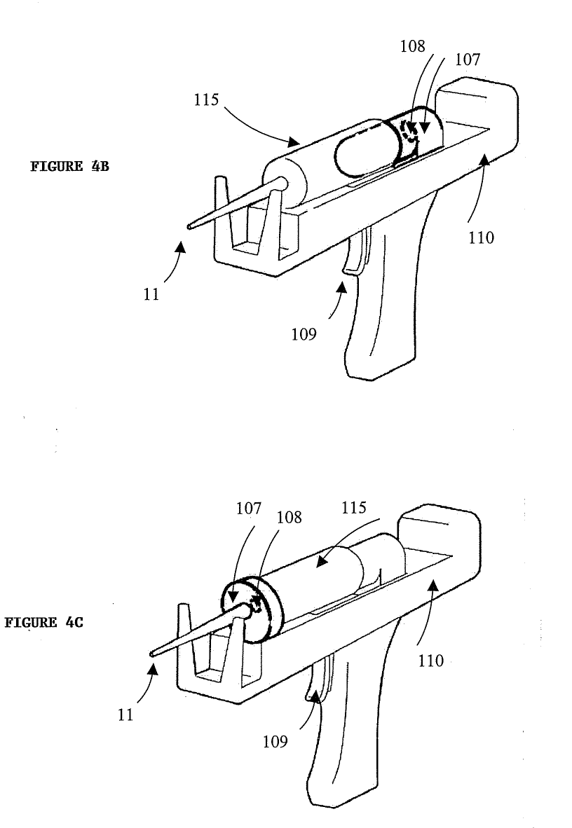

The injecting means 115 is adapted to contain liquid to be injected to a tissue, preferably to a target area in said tissue, and to inject a predetermined volume of said liquid into a predetermined depth of the tissue through a needle 11. The injecting means 115 may be a syringe or syringe like container and it is functionally connected to the heating means 107 and to the temperature sensor 108. The heating means 107 may have various shapes and may be located in different positions relative to the injecting means. Optional shapes and locations of the heating means are depicted in FIG. 4 and will be described in more details henceforth. The injecting means 115 is also functionally connected to the electromechanical element 110 that physically activate the injecting means, a needle attached thereto, or both, and moves them toward the tissue to perform the injection.

Generally, the delivery of heat to a tissue according to the present invention is conducted by injection of a preheated liquid to said tissue. For that purpose any inert liquid that is adapted for injection is suitable for the methods and apparatus of the invention. Examples of such liquids are water, water for injection, and physiological solution like Ringer solution. The liquid is heated before or during the injection to a predetermined temperature level, preferably, in the range of 45° C.-75° C. The predetermined temperature is determined according to different parameters including without limiting: the depth of injection, the injection volume, the tissue type, the type of manipulation required, and combinations thereof. The physical size of the treated subject may also affect the predetermined values of the injected liquid. The heating of the liquid to a predetermined temperature level may be performed inside the injecting means (FIG. 4B), or by heating the injecting means with the liquid (FIG. 4A). In such embodiments, the heating of the liquid is conducted before the injection of the liquid. In a different embodiment, the heating of the liquid is performed in a connector (FIG. 4C) that is functionally connecting the injecting means and the needle. In such embodiment the heating of the liquid is conducted during the injection.

The apparatus provided herein enables to direct the injection of the heated liquid to a specific layer of a tissue, and to a target area in the tissue by controlling the depth of injection and the injection volume. In specific embodiments, the manipulation of a tissue is conducted by the injection of the heated liquid to the subcutaneous layer of the skin, to the fat layer or to a cancerous tissue.

In one another embodiment of the invention, the MesoTherm apparatus 100 comprises an electromechanical element 110 as an integrated part of the apparatus. In such embodiment, the controller of the MCU 104 further includes an appropriate control circuitry adapted to activate or deactivate the electromechanical element 110 according to the sensed temperature of the heated liquid. When the temperature level of the liquid reaches the predetermined value, the controller activates an injection trigger 109 that functionally operates the electromechanical element 110 to thereby move the injecting means 115 or a needle 11 attached thereto, or both, toward the tissue and inject the heated liquid. However, if the temperature level of the liquid for injection has not reached the predetermined temperature level or it has exceeded the predetermined value, the controller will block the injection of the heated liquid. The injection velocity may also very according to the tissue type, the manipulation requires, the operator preferences and the tolerance of the treated object. In general, the pain level is reduced inversely to the injection velocity, i.e. as far as the injection velocity is higher the pain level is lower.

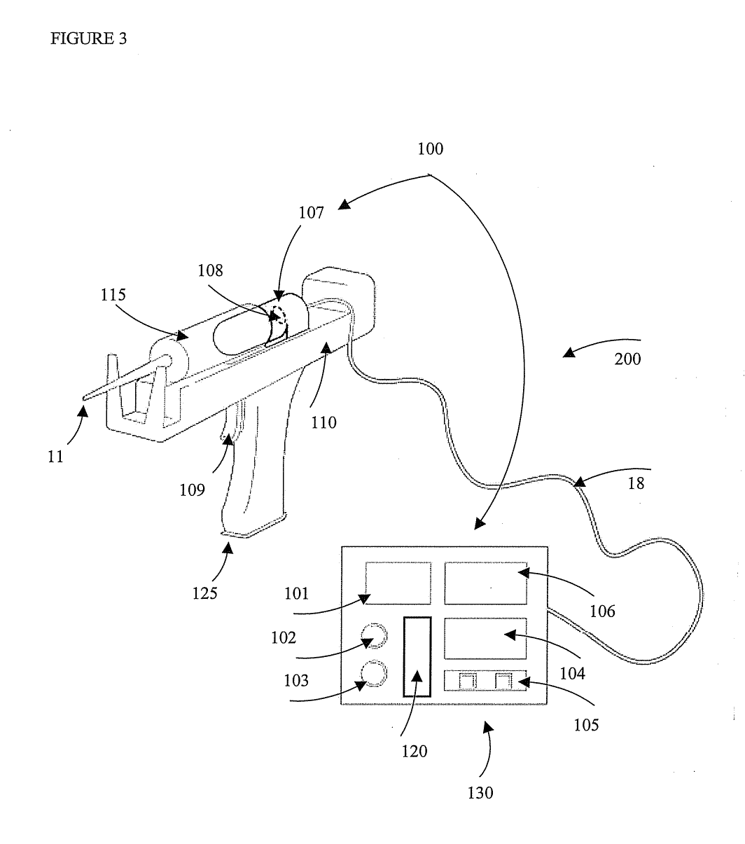

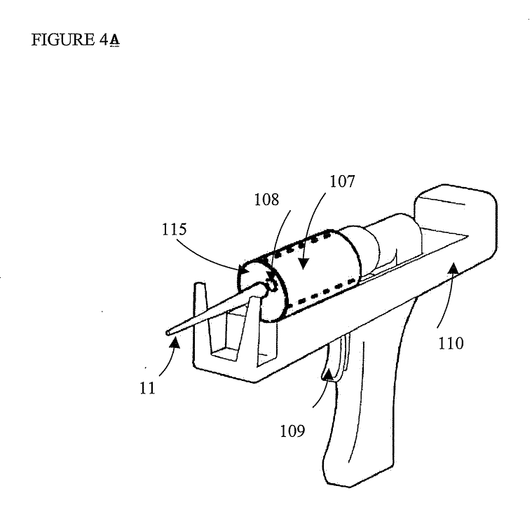

In accordance with one embodiment of the invention, the injection trigger 109 is an on/off switch adapted to enable the activation or the deactivation of the electromechanical element. More specifically, by turning the injection trigger switch on, the electromechanical element 110 becomes functionally accessible to receive signals from the MCU 104 and to move the injecting means 115 or the needle attached thereto, or both, toward the tissue and inject the heated liquid. In yet, a different embodiment of the invention (shown in FIG. 3), the MesoTherm apparatus 100 does not comprise an electromechanical element as an integral part of it and is functionally assembled with an independent electromechanical device 125. In such an embodiment, the injection trigger 109 may be a mechanical switch that controls the activation and deactivation of the electromechanical element 110. When the injection switch 109 is set on an “off” mode the electromechanical element is unable to move the injecting means 115 toward the tissue and inject the liquid. However, when the injection switch 109 is set on an “on” mode it allows the electromechanical element 110 to function. In additional embodiment of the present invention pulling the injection trigger activate the electromechanical element and leads to injection. In a further embodiment, the electromechanical device 125 comprises a safe-action trigger system, based, for example on a sub-trigger mechanism or on a safety lever protruding through the front face of the trigger itself. In such an embodiment, when the trigger finger depresses the trigger system, the safety lever is contacted first and is depressed until it becomes flush with the face of the trigger, thereby unlocking the trigger so that it may be pulled to the rear in order for the electromechanical device to inject. In general, the electromechanical element in accordance with the present invention may be consisted of a pneumatic mechanism, a hydraulic mechanism or any other mechanism known in the art that is suitable for the purposes of the apparatus presented herein.

In yet a further embodiment, the targeted delivery of heat to said tissue by the MesoTherm apparatus may be performed by a serial of subsequent injections. In such an embodiment, between each subsequent injections, the liquid remained in the needle after the injection, is drawn back into the injecting means. The subsequent injections may be performed in a random manner or according to a predetermined pattern. The pattern may employ marking at least one of: the depth of injection, the distance between adjacent injecting points, the injection volume, and combinations thereof.

Generally, directing the injecting means 115 to the tissue may employ physical markers or virtual markers or combination of both, on the tissue. In such scenario, the physical markers are at least partly adhered to the tissue.

The MesoTherm apparatus 100 may further comprise a display means 106 a speaker means 120, or both. In such embodiment, the display means and/or the speaker means are functionally connected to the MCU 104. The display means is adapted to display predefined features, such as the temperature level of the heated liquid, the depth of injection, the injection volume, the injection velocity, the stage of the manipulation process and different written alerting notices such as: “SYSTEM READY”, “OVER HEAT”, “BELOW TEMPERATURE”, “BATTERY LOW”, “PRESS ON TRIGGER”, “COOLING DOWN”, etc. The display means may also comprise an emitting light in at least two different colors, where each color indicates a different feature. In a specific embodiment the display means 106 includes a two-color light emitting diode that provides the user a simple indication on the status of the manipulation process, or indications on a predetermined feature. For example, red light may indicate that the temperature level of the liquid contained in the injecting means 115 is not equal to the predetermined temperature level and thus, the MesoTherm apparatus is not ready for injection, while green light may indicate that the temperature level of the liquid has reached the predetermined temperature level and the MesoTherm apparatus is ready for injection. The speaker means 120 is adapted to produce at least one vocal alert relating to the predetermined features mentioned above, or relating to the status of the apparatus. The vocal alert may be a vocal notice similar to the written notices denoted above, a beep or an alarm. The apparatus may comprise different kind of sounds, wherein each sound is indicative of a specific feature. For example, when the apparatus is ready for injection the speaker means produces a specific sound informing the operator that the apparatus is ready, when the temperature is too low the speaker means produces a sound that is indicative of low temperature, when the temperature is too high, the speaker means produces an alarm and a vocal notice such as “OVER HEAT”, and so on. The apparatus may further comprise a self test circle and an auto shut down mechanism.

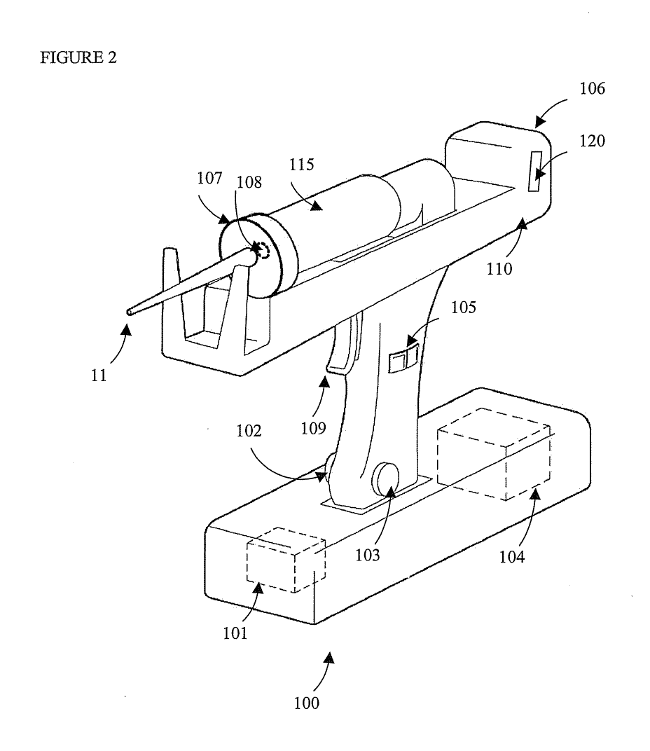

Reference is now made to FIG. 2 which is a simplified pictorial illustration of a MesoTherm apparatus constructed in accordance with a preferred embodiment of the present invention, in which the electromechanical element 110, is an integral part of the apparatus and is functionally connected to the injection trigger 109 and to the injecting means 115. The apparatus depicted in FIG. 2 is generally adapted for manipulating a tissue by targeted delivery of heat and comprises: a power supply 101, an apparatus on/off switch 102, an MCU 104 comprising a controller adapted to monitor the temperature sensor signal and to activate or deactivate the heating means 105 and the electromechanical element 110 according to the temperature sensed, an input unit 105, a temperature on/off switch 103, a heating means 105 adapted to heat the liquid contained in the injecting means 115 to a predetermined temperature level, a temperature sensor 108 adapted to identify the temperature level of the liquid contained in the injecting means 115 and to produce a signal indicating said temperature, an injection trigger (on/off switch) 109, an injecting means 115 adapted to contain liquid to be injected to a tissue, preferably to a target area in said tissue and to inject a predetermined volume of said liquid into a predetermined depth of the tissue through a needle 11, an electromechanical element 110 functionally connected to the injection trigger 109 and to the injecting means 115 and adapted to allow the injection of the heated liquid, a display means 106, and a speaker means 120.

Reference is now made to FIG. 3, which is a simplified pictorial illustration of a system for aesthetic and medical treatment 200 of a person in need of such treatment according to a preferred embodiment of the invention. The system 200 comprises a MesoTherm apparatus 100 adapted for manipulating a tissue by targeted delivery of heat into said tissue according to any of the embodiments described herein that do not comprise an electromechanical element and an injection trigger as integral parts thereof, and an electromechanical device 125 adapted for assembling with the MesoTherm apparatus 100 to thereby allow the delivery of heat to the tissue according to a predetermine pattern. In accordance with a specific embodiment depicted in FIG. 3 the MesoTherm apparatus comprises: a heating means 107 adapted to heat a liquid contained within an injecting means 115 to a predetermined temperature level; an injecting means 115 adapted to contain liquid to be injected to the tissue, preferably to a target area in said tissue, and to inject a predetermined volume of said liquid into a predetermined depth of said tissue through a needle 11; a temperature sensor 108 adapted to identify the temperature level of the liquid within the injecting means and to produce a signal indicating said temperature level; an MCU 104 comprising a controller adapted to monitor the temperature sensor signal and to activate the heating means 107 upon said controller determining that the temperature level of the liquid is below the predetermined temperature, or to deactivate the heating means upon said controller determining that the temperature level of the liquid is equal to or above the predetermined temperature level; a power supply unit 101; an apparatus on/off switch 102; an input unit 105; a temperature on/off switch 103, a display means 106, and a speaker 120. In the specific embodiment depicted in FIG. 3 the power supply unit 101, the MCU 104, the apparatus on/off switch 102, the input unit 105, the temperature on/off switch 103, the display means 106 and the speaker 120 are incorporated into a housing unit 130 and are functionally connected to the heating means 107, the temperature sensor 108 and the injecting means 115 by a connecting means 18. The connecting means 18 may be any cord known in the art that is adapted for delivering signals between the different units of the apparatus. In one another embodiment the connection between the apparatus units that are gathered in the housing unit 130 and the other units of the apparatus is a wireless connection. The wireless connection in such embodiment may be any wireless techniques that is know in the art.

The electromechanical device 125 is an independent device, which comprises at least an electromechanical element 110 and an injection trigger 109 adapted to activate the electromechanical element. In such embodiment, upon pressing on the injection trigger 109, at least part of the injecting means, a needle attached to it, or both, moves toward the tissue, the needle penetrates the tissue to a predefined depth, in a predetermined velocity, and a predefined volume of the heated liquid is injected to the tissue.

The independent electromechanical device 125 may be any device known in the art that is functionally capable of assembling with the MesoTherm apparatus 100 of the invention. Examples of such suitable electromechanical devices that are available on the market and know generally as the Mesogun, injection gun, mesotherapy gun or pistolets are the fully automatic devices such as the U225 Mesogun; the Pistor 4, the Mesomega, the DHN 4, the DHN3, the DHN2, the DHN1, the Meso-system; and the semi automated devices such as the Dermotherap, the Meso-basic and the Den'hub (additional information on mesotherapy guns is available on http://www.usameso.com/mesogun.php). The system depicted in FIG. 3 may further comprise a self test circle and an auto shut down mechanism.

Reference is now made to FIGS. 4A-4C, which schematically illustrate various shapes and locations of the heating means 107 and the temperature sensor 108 relative to the injecting means 115. FIG. 4A represent an embodiment in which the heating means is coating the inner wall of the injecting means 115 that contains the liquid for injection. In this specific embodiment the temperature sensor 108 is positioned on the anterior wall of the injecting means adjacent to the connection cavity of the injecting means 115 and the needle 11. The sensor may also be attached to the piston of the injecting means (not shown); FIG. 4B describes additional embodiment in which the heating means 107 is located inside the injecting means 115 and it is physically separated from the injecting means. The temperature sensor is positioned near the heating means 115, and both are covered by the liquid for injection; FIG. 4C illustrates one additional embodiment in which the heating means 107 is located outside the injecting means 115 and functionally connecting the injecting means 115 and the needle 11. According to this embodiment, the heating means is designed as a connector, and therefore the heating of the liquid is conducted during the injection process. The temperature sensor 108 is located in such embodiment, on the anterior side of the heating means adjacent to the needle 11.

The present invention also provides a method for manipulating a tissue by targeted delivery of heat, which comprises the steps of:

a. heating a liquid contained in an injecting means that is functionally connected to an electromechanical element to a predetermined temperature level;

b. directing the injecting means to the tissue; and

c. activating the electromechanical element to functionally operate the injecting means to inject a predetermined volume of the heated liquid into a predetermined depth of the tissue through a needle attached to said injecting means, to thereby manipulate the tissue in the injected area.

Activating the electromechanical element 110 according to this method is conducted by pressing on the injection trigger 109. Upon pressing on the injection trigger at least part of the injecting means 115, a needle 11 attached to it, or both moves toward the tissue, to thereby inject the heated liquid to the tissue.

Manipulating a tissue according to the present invention includes without limitation: fat reduction, cellulite refinement, body contouring, skin tightening, hair re-growth, wrinkle elimination, scar improvement, acne treatment, stretch marks improvement, destruction of cancer cells and necrosis induction.

The delivery of heat to a tissue is conducted by injection of a preheated liquid. For that purpose any inert liquid that is adapted for injection is suitable for the methods of the invention. Examples of such liquids are water, water for injection, and physiological solution like Ringer solution. The liquid in heated to predetermined temperature level, preferably, in the range of 45° C.-75° C. The predetermined temperature is determined according to different parameters including without limiting: the depth of injection, the injection volume, the tissue type, the type of manipulation required, and combinations thereof. The physical size of the treated subject may also affect the predetermined values of the injected liquid. The heating of the liquid to a predetermined temperature level may be performed inside the injecting means (FIG. 4B), or by heating the injecting means with the liquid (FIG. 4A). In such embodiments, the heating of the liquid is conducted before the injection of the liquid. In a different embodiment, the heating of the liquid is performed in a connector (FIG. 4C) that is functionally connecting the injecting means and the needle. In such embodiment the heating of the liquid is conducted during the injection.

The methods provided herein enables to direct the injection of the heated liquid to a specific layer of a tissue, i.e. to a target area in the tissue, by controlling the depth of injection and the injection volume. In specific embodiments, the manipulation of a tissue is conducted by the injection of the heated liquid to the subcutaneous layer of the skin, to a fat layer or to a cancerous tissue.

In yet a further embodiment, the targeted delivery of heat to said tissue may be performed by a serial of subsequent injections. In such an embodiment, between each subsequent injections, the liquid remained in the needle after the injection, is drawn back into the injecting means. The subsequent injections may be performed in a random manner or according to a predetermined pattern. The pattern may employ marking at least one of: the depth of injection, the distance between adjacent injecting points, the injection volume, and combinations thereof.

Generally, directing the injecting means to the tissue may employ physical markers or virtual markers, or combination of both, on the tissue. In such scenario, the physical markers are at least partly adhered to the tissue.

The methods provided herein may further comprise a step of sensing the temperature level of the liquid contained in the injecting means and producing a signal indicating said temperature level. In additional embodiment, the methods comprise a further step of deactivating the heating means upon determining that the temperature level of the liquid contained in the injecting means is equal to or above the predetermined temperature level, and activating the heating means upon determining that the temperature level of the liquid contained in the injecting means is below the predetermined temperature level.

In an additional embodiment of the invention, the methods provided herein also comprise a step of displaying predefined features on a display means 106, and/or producing at least one vocal alert by a speaker means 120. The displayed featured include any relevant parameter to the operator, such as the temperature level of the heated liquid, the depth of injection, the injection volume, the injection velocity, the stage of the process, and different written alerting notices such as: “SYSTEM READY”, “OVER HEAT”, “BELOW TEMPERATURE”, “BATTERY LOW”, “PRESS ON TRIGGER”, COOLING DOWN”, etc. The display means may also comprise an emitting light in at least two different colors, where each color indicates a different feature or stage of the method. The speaker means is adapted to produce at least one vocal alert, related to the features mentioned above, or relating to the status of the process. The vocal alert may be a vocal notice similar to the written notices denoted above, a beep or an alarm. The speaker means may produce different kind of sounds, wherein each sound is indicative of a specific feature. For example, when the apparatus or the system are ready for injection it produce a specific sound informing the operator that the apparatus is ready, when the temperature is too low the speaker means produce a sound that is indicative of low temperature, when the temperature is too high, the speaker means produce an alarm and a vocal notice such as “OVER HEAT”, and so on. In a specific embodiment, the operator is able to select a preferred type of sound from a sound menu.

The present invention further provides a method for aesthetic and medical treatment comprising the steps of:

a. heating a liquid contained in an injecting means that is functionally assembled with an electromechanical device to a predetermined temperature level;

b. directing the injecting means to said tissue; and

c. activating the electromechanical device to functionally operate the injecting means and inject a predetermined volume of said heated liquid into a predetermined depth of the tissue through a needle functionally attached to the injecting means.

The aesthetic and medical treatments includes without limitation: fat reduction, cellulite refinement, body contouring, skin tightening, hair re-growth, wrinkle elimination, scar improvement, acne treatment, stretch marks improvement, destruction of cancer cells and necrosis induction. The delivery of heat to the tissue is generally conducted in the same manner as described for the methods above by injection of a preheated inert liquid adapted for injection, such as water, water for injection, and physiological solution like Ringer solution, to the tissue, and preferably to a target area in said tissue. The liquid is heated to a predetermined temperature level, preferably, in the range of 45° C.-75° C. The predetermined temperature level is determined according to different parameters including without limiting: the depth of injection, the injection volume, the tissue type, the type of manipulation required, and combinations thereof.

The electromechanical device 125 comprises at least an electromechanical element 110 and an injection trigger 109 adapted to activate an electromechanical element. In a specific embodiment, the activation of the electromechanical device includes pressing on the injection trigger to thereby move at least part of the injecting means or the needle attached thereto, or both, toward the tissue, and inject the heated liquid to the tissue. The injection of the heated liquid to the tissue is functionally controlled by the temperature level of the liquid.

The heating of the liquid to a predetermined temperature level may be performed inside the injecting means or by heating the injecting means with the liquid as described above. In such embodiments, the heating of the liquid is conducted before the injection of the liquid. In a different embodiment, the heating of the liquid is performed in a connector that is functionally connecting the injecting means and the needle. In such an embodiment the heating of the liquid is conducted during the injection. The injection of the heated liquid may be directed to a specific layer of the tissue, i.e. to a target area of the tissue, by controlling the depth of injection and the injection volume. In specific embodiments, the manipulation of a tissue is conducted by the injection of the heated liquid to the subcutaneous layer of the skin, to a fat layer or to a cancerous tissue. Generally, directing the injecting means to the tissue may employ physical markers or virtual markers or combination of both, on the tissue. In such scenario, the physical markers are at least partly adhered to the tissue.

In yet a further embodiment, the targeted delivery of heat to said tissue may be performed by a serial of subsequent injections. In such an embodiment, between each subsequent injections, the liquid remained in the needle after the injection, is drawn back into the injecting means. The subsequent injections may be performed in a random manner or according to a predetermined pattern. The pattern may employ marking at least one of: the depth of injection, the distance between adjacent injecting points, the injection volume, and combinations thereof.

The method provided herein may further comprise a step of sensing the temperature level of the liquid contained in the injecting means and producing a signal indicating said temperature level. In additional embodiment, the method comprises a further step of deactivating the heating means upon determining that the temperature level of the liquid contained in the injecting means is equal to or above the predetermined temperature level, and activating the heating means upon determining that the temperature level of the liquid contained in the injecting means is below the predetermined temperature level.

In an additional embodiment of the invention, the method for aesthetic and medical treatment provided herein, further comprise a step of displaying predefined features on a display means 106, and/or producing at least one vocal alert by a speaker means 120. The displayed featured include any relevant parameter to the operator, such as the temperature level of the heated liquid, the depth of injection, the injection volume, the injection velocity, the stage of the process, and different written alerting notices as mentioned above. The display means may also comprise an emitting light in at least two different colors, where each color indicates a different feature or stage of the process. The vocal alert may be an alarm, a beep or different vocal alerting notices such as the written noticed described above.

1. A method for manipulating a tissue by targeted delivery of heat, said method comprising:
a. heating a liquid contained in an injecting means to a predetermined temperature level;
b. directing the injecting means to said tissue; and
c. injecting a predetermined volume of said heated liquid into a predetermined depth of the tissue through a needle attached to said injecting means, to thereby manipulate the tissue in the injected area.

2. A method according to claim 1, wherein said manipulating is selected from the group consisting of: fat reduction, cellulite refinement, body contouring, skin tightening, hair re-growth, wrinkle elimination, scar improvement, acne treatment, stretch marks improvement, destruction of cancer cells and necrosis induction.

3. A method according to claim 1, wherein said liquid is an inert liquid adapted for injection, and said predetermined temperature level is in the range of 45° C.-75° C.

4. (canceled)

5. A method according to claim 1, wherein said predetermined temperature level is determined according to at least one of the parameters selected from the group consisting of: the depth of injection, the injection volume, the tissue type, the type of manipulation required, and combinations thereof.

6. A method according to claim 1, wherein the heating of said liquid to a predetermined temperature level is performed either inside the injecting means, or by heating the injecting means with the liquid, or in a connector, said connector is functionally connecting the injecting means and the needle.

7. (canceled)

8. (canceled)

9. A method according to claim 1, wherein the targeted delivery of heat to said tissue is performed by a serial of subsequent injections.

10. A method according to claim 9, wherein said subsequent injections are performed according to a predetermined pattern, said pattern employs marking at least one of: the depth of injection, the distance between adjacent injecting points, the injection volume, and combinations thereof.

11. (canceled)

12. A method according to claim 9, wherein between each subsequent injection, the liquid remained in the needle after the injection is drawn back into the injecting means.

13. A method according to claim 1, wherein said directing the injecting means employs either physical markers or virtual markers or combinations thereof, on the tissue, and wherein said physical markers are at least partly adhered to the tissue.

14. (canceled)

15. A method according to claim 6, further comprising sensing the temperature of the liquid contained in the injecting means or in the connector and producing a signal indicating said temperature.

16. A method according to claim 15, further comprising deactivating the heating means upon determining that the temperature level of the liquid contained in the injecting means or in the connector is equal to or above the predetermined temperature level, and activating the heating means upon determining that the temperature level of the liquid contained in the injecting means or in the connector is below the predetermined temperature level.

17. A method according to claim 1, further comprising displaying predefined features on a display means.

18. A method according to claim 17, wherein said features are selected from the group consisting of: temperature level, depth of injection, injection volume, injection velocity, stage of process and alerting notices.

19. A method according to claim 17, wherein said displaying comprises emitting light in at least two different colors, and wherein each color indicates different feature or stage of the process.

20. A method according to claim 1, further comprising producing at least one vocal alert by a speaker means.

21. An apparatus adapted for manipulating a tissue by targeted delivery of heat, said apparatus comprising:
A heating means adapted to heat a liquid contained in an injecting means to a predetermined temperature level;
An injecting means adapted to contain liquid to be injected to a tissue and to inject a predetermined volume of said liquid into a predetermined depth of said tissue through a needle;
A temperature sensor adapted to identify the temperature level of the liquid contained in the injecting means and to produce a signal indicating said temperature level;
A controller adapted to monitor the sensor signal and to activate the heating means upon said controller determining that the temperature level of the liquid is below the predetermined temperature level or to deactivate the heating means upon said controller determining that the temperature level of the liquid is equal to or above the predetermined temperature level.

22. An apparatus according to claim 21, wherein said manipulating is selected from the group consisting of: fat reduction, cellulite refinement, body contouring, skin tightening, hair re-growth, wrinkle elimination, scar improvement, acne treatment, stretch marks improvement, destruction of cancer cells ad necrosis induction.

23. An apparatus according to claim 21, wherein said liquid is an inert liquid adapted for injection, and said predetermined temperature level is in the range of 45° C.-75° C.

24. (canceled)

25. An apparatus according to claim 21, wherein said predetermined temperature level is determined according to at least one of the parameters selected from the group consisting of: the depth of injection, the injection volume, the tissue type, the type of manipulation required, and combinations thereof.

26. (canceled)

27. An apparatus according to claim 21, further comprising a connector, said connector is functionally connecting the injecting means and the needle, and wherein the heating of said liquid to a predetermined temperature level is performed in the connector.

28. (canceled)

29. An apparatus according to claim 21, wherein the targeted delivery of heat to said tissue is performed by a serial of subsequent injections.

30. An apparatus according to claim 29, wherein between each subsequent injection, the liquid remained in the needle after the injection is drawn back into the injecting moans.

31. An apparatus according to claim 29, wherein said subsequent injections are performed according to a predetermined pattern, said pattern employs marking at least one of the depth of injection the distance between adjacent injecting points the injection volume, and combinations thereof.

32. (canceled)

33. An apparatus according to claim 23, wherein said pattern employs either physical Or virtual markers, or combinations thereof, on the tissue, and wherein said physical markers are at least partly adhered to the tissue.

34. (canceled)

35. An apparatus according to claim 21, further comprising a display means adapted to displaying predefined features.

36. An apparatus according to claim 35, wherein said features are selected from the group consisting of: temperature level, depth of injection, injection volume, injection velocity, stage of the process, and alerting notices.

37. An apparatus according to claim 35, wherein said display means are adapted to emit light in at least two different colors, and wherein each color indicates a different mode or status of the apparatus.

38. An apparatus according to claim 21, further comprising a speaker means adapted to produce at least one vocal alert.

39. (canceled)

40. A system for aesthetic and medical treatment of a person in need of such treatment, said system comprising:
a. An apparatus adapted for manipulating a tissue by targeted delivery of heat into said tissue; and
b. An electromechanical device adapted for assembling with said apparatus, and allowing the delivery of heat to said tissue according to a predetermined pattern.

41. A system according to claim 40, wherein said apparatus comprises at least a heating means, an injecting means, a temperature sensor and a controller, and wherein the electromechanical device comprises at least an electromechanical element and an injection trigger adapted to activate said electromechanical element.

42. A system according to claim 41, wherein upon pressing on the injection trigger at least part of the injecting means, a needle attached thereto, or both, moves toward the tissue, to thereby inject the heated liquid to said tissue.

43. (canceled)

44. A system according to claim 40, wherein said aesthetic and medical treatment is selected from the group consisting of: fat reduction, cellulite refinement, body contouring, skin tightening, hair re-growth, wrinkle elimination, scar improvement, acne treatment, stretch marks improvement, destruction of cancer cells and necrosis induction.

45. A system according to claim 40, wherein said targeted delivery of heat into a tissue is conducted by injection of an inert liquid in a predetermined temperature level to said tissue.

46. A system according to claim 45, wherein said predetermined temperature level is in the range of 45° C.-75° C.

47. A system according to claim 45, wherein said predetermined temperature level is determined according to at least one of the parameters selected from the group consisting of: the depth of injection, the injection volume, the tissue type, the type of manipulation required, and combinations thereof.

48. A system according to claim 45, wherein the beating of said liquid to a predetermined temperature is performed either inside be injecting means, or by heating the injecting means with the liquid or in a connector, said connector is functionally connecting the injecting means and a needle.

49. (canceled)

50. (canceled)

51. A system according to claim 40, wherein the targeted delivery of heat to said tissue is performed by a serial of subsequent injections.

52. A system according to claim 51, wherein between each subsequent injection, the liquid remained in the needle after the injection is drawn back into the injecting means.

53. A system according to claim 51, wherein said subsequent injections are performed according to a predetermined pattern, said pattern employs marking at least one of: the depth of injection, the injection volume the distance between adjacent injecting points and combinations thereof.

54. (canceled)

55. A system according to claim 39, wherein said predetermined pattern employs physical markers or virtual markers or combinations thereof, on the tissue, and wherein said physical markers are at least partly adhered to the tissue.

56. (canceled)

57. A system according to claim 48, wherein said temperature sensor is adapted to sense the temperature level of the liquid contained in the injecting means or in the connector and to produce a signal indicating said temperature.

58. A system according to claim 48, wherein said controller is adapted to deactivate the heating means upon determining that the temperature level of the liquid contained in the injecting means or in the connector is equal to or above the predetermined temperature level and to activate the heating means upon determining that the temperature level of the liquid contained in the injecting means or in the connector is below the predetermined temperature level.

59. A system according to claim 41, further comprising a display means adapted to display predefined features.

60. (canceled)

61. A system according to claim 59, wherein said display means comprises emitting light in at least two different colors, and wherein each color indicates a different mode or status of the system.

62. A system according to claim 41, further comprising a speaker means adapted to produce at least one vocal alert.

63. A system for aesthetic and medical treatment comprising:
a. An apparatus adapted for manipulating a tissue by targeted delivery of heat into the tissue according to claim 21; and
b. An electromechanical device adapted for assembling with said apparatus, and allowing the delivery of heat to said tissue according to a predetermined pattern.

64. A method for aesthetic and medical treatment comprising:
a. heating a liquid contained in an injecting means that is functionally assembled with an electromechanical device to a predetermined temperature level;
b. directing the injecting means to said tissue; and
c. activating said electromechanical device to functionally operate the injecting means and allow the injection of a predetermined volume of said heated liquid into a predetermined depth of the tissue through a needle functionally attached to the injecting means.

65. A method for aesthetic and medical treatment according to claim 64, wherein the electromechanical device comprises at least an injection trigger adapted to activate an electromechanical element.

66. A method for aesthetic and medical treatment according to claim 64, wherein activating the electromechanical device includes pressing on said injection trigger to thereby move at least part of the injecting means, a needle attached thereto, or both, toward the tissue, to thereby inject the heated liquid to said tissue.

67. A method for aesthetic and medical treatment according to claim 64, wherein the injection of said heated liquid to the tissue is functionally controlled by the temperature level of said liquid.

68. A method according to claim 64, wherein said aesthetic and medical treatment is selected from the group consisting of: fat reduction, cellulite refinement, body contouring, skin tightening, hair re-growth, wrinkle elimination, scar improvement, acne treatment, stretch marks improvement, destruction of cancer cells and necrosis induction.

69. A method according to claim 64, wherein said liquid is an inert liquid adapted for injection and said predetermined temperature level is in the range of 45° C.-75° C.

70. (canceled)

71. A method according to claim 64, wherein said predetermined temperature level is determined according to at least one of the parameters selected from the group consisting of: the depth of injection, the injection volume, the tissue type, the type of manipulation required, and combinations thereof.

72. A method according to claim 64, wherein the heating of said liquid to a predetermined temperature level is performed either inside the injecting means, or by heating the injecting means with the liquid, or in a connector, said connector is functionally connecting the injecting means and the needle.

73. (canceled)

74. (canceled)

75. A method according to claim 64, wherein the targeted delivery of heat to said tissue is performed by a serial of subsequent injections.

76. (canceled)

77. (canceled)

78. A method according to claim 75, wherein between each subsequent injection, the liquid remained in the needle after the injection is drawn back into the injecting means.

79. A method according to claim 64, wherein said directing the injecting means employs either physical or virtual markers or combinations thereof on the tissue, and wherein said physical markers are at least partly adhered to the tissue.

80. (canceled)

81. A method according to claim 72, further comprising sensing the temperature level of the liquid contained in the injecting means or in the connector and producing a signal indicating said temperature level.

82. A method according to claim 72, further comprising deactivating the heating means upon determining that the temperature level of the liquid contained in the injecting means or in the connector is equal to or above the predetermined temperature level and activating the heating means upon determining that the temperature level of the liquid contained in the in injecting means or in the connector is below the predetermined temperature level.

83. A method according to claim 64, further comprising displaying predetermined features on a display means, said features are selected from the group consisting of: temperature level, depth of injection, injection volume, injection velocity, stage of the process and alerting notices.

84. (canceled)

85. A method according to claim 64, further comprising producing at least one vocal alert by a speaker means.

86. An apparatus adapted for manipulating a tissue by targeted delivery of heat, said apparatus comprising:
A heating means adapted to heat a liquid contained in an injecting means to a predetermined temperature level;
An injecting means adapted to contain liquid to be injected said tissue and to inject a predetermined volume of said liquid into a predetermined depth of the tissue through a needle;
A temperature sensor adapted to identify the temperature level of the liquid contained in the injecting means and to produce a signal indicating said temperature level;
A controller adapted to monitor the sensor signal and to activate or deactivate said heating means and an electromechanical element according to the temperature sensed.
An electromechanical element adapted to allow the injection of said heated liquid.

87. An apparatus according to claim 86, wherein said electromechanical element is functionally connected to an injection trigger and to said injecting means.

88. An apparatus according to claim 86, wherein said manipulating is selected from the group consisting of: fat reduction, cellulite refinement, body contouring, skin tightening, hair re-growth, wrinkle elimination, scar improvement, acne treatment, stretch marks improvement, destruction of cancer cells and necrosis induction.

89. An apparatus according to claim 86, wherein said liquid is an inert liquid adapted for injection and said predetermined temperature level is in the range of 45° C.-75° C.

90. (canceled)

91. An apparatus according to claim 86, wherein said predetermined temperature level is determined according to at least one of the parameters selected from the group consisting of: the depth of injection, the injection volume, the tissue type, the type of manipulation required, and combinations thereof.

92. (canceled)

93. An apparatus according to claim 86, further comprising a connector, said connector is functionally connecting the injecting means and the needle, and wherein the heating of said liquid to a predetermined temperature level is performed in the connector.

94. (canceled)

95. An apparatus according to claim 86, wherein the targeted delivery of heat to said tissue is performed by a serial of subsequent injections.

96. An apparatus according to claim 95, wherein between each subsequent injection, the liquid remained in the needle after the injection is drawn back into the injecting means.

97. (canceled)

98. (canceled)

99. (canceled)

100. (canceled)

101. An apparatus according to claim 86, further comprising a display means adapted to displaying predefined features.

102. (canceled)

103. An apparatus according to claim 101, wherein said display means are adapted to emit light in at least two different colors, and wherein each color indicates a different mode or status of the apparatus.

104. An apparatus according to claim 86, further comprising a speaker means adapted to produce at least one vocal alert.

105. (canceled)

106. An apparatus according to claim 87, wherein upon pressing on said injection trigger at least part of the injecting means, a needle attached thereto, or both, moves toward the tissue, to thereby inject the heated liquid to said tissue.

107. A method for manipulating a tissue by targeted delivery of heat, said method comprising:
a. heating a liquid contained in an injecting means that is functionally connected to an electromechanical element to a predetermined temperature level;
b. directing the injecting means to said tissue; and
c. activating said electromechanical element to functionally operate the injecting meals to inject a predetermined volume of said heated liquid into a predetermined depth of the tissue through a needle attached to said injecting means, to thereby manipulate the tissue in the injected area.

108. A method according to claim 107, wherein said activating comprises pressing on the injection trigger, and wherein upon pressing on said injection trigger at least part of the injecting means, a needle attached thereto, or both moves toward the tissue, to thereby inject the heated liquid to said tissue.

109. A method according to claim 107, wherein said manipulating is selected from the group consisting of: fat reduction, cellulite refinement, body contouring, skin tightening, hair re-growth, wrinkle elimination, scar improvement, ache treatment, stretch marks improvement, destruction of cancer cells and necrosis induction.

110. A method according to claim 107, wherein said liquid is an inert liquid, adapted for injection, and said predetermined temperature level is in the range of 45° C.-75° C.

111. (canceled)

112. A method according to claim 107, wherein said predetermined temperature level is determined according to at least one of the parameters selected from the group consisting of: the depth of injection, the injection volume, the tissue type, the type of manipulation required, and combinations thereof.

113. A method according to claim 107, wherein the heating of said liquid to a predetermined temperature level is performed either inside the injecting means, or by heating the injecting means with the liquid, or in a connector, said connector is functionally connecting the injecting means and the needle.

114. (canceled)

115. (canceled)

116. A method according to claim 107, wherein the targeted delivery of heat to said tissue is performed by a serial of subsequent injections.

117. A method according to claim 116, wherein between each subsequent injection, the liquid remained in the needle after the injection is drawn back into the injecting means.

118. (canceled)

119. (canceled)

120. A method according to claim 112, wherein said directing the injecting means employs either physical markers or virtual markers; or combinations thereof on the tissue, and wherein said physical markers are at least partly adhered to the tissue.

121. (canceled)

122. A method according to claim 113, further comprising sensing the temperature level of the liquid contained in the injecting means or in the connector and producing a signal indicating said temperature level.

123. A method according to claim 122, further comprising deactivating the heating means upon determining that the temperature level of the liquid contained in the injecting means or in the connector is equal to or above the predetermined temperature level and activating the heating means upon determining that the temperature level of the liquid contained in the injecting means or in the connector is below the predetermined temperature level.

124. A method according to claim 113, further comprising deactivating the electromechanical element upon determining that the temperature level of the liquid contained in the injecting means or in the connector is below or above the predetermined temperature level and activating the electromechanical element upon determining that the temperature level of the liquid contained in the injecting means or in the connector is equal to the predetermined temperature level.

125. A method according to claim 112, further comprising displaying predefined features on a display means, said features are selected from the group consisting of: temperature level, depth of injection, injection volume injection velocity, stage of the process and alerting notices.

126. (canceled)

127. A method according to claim 125, wherein said displaying comprises emitting light in at least two different colors, and wherein each color indicates a different feature or stage of the process.

128. A method according to claim 112, further comprising producing at least one vocal alert by a speaker means.
